(19) United States
(12) Patent Application Publication
(10) Pub. No.: US 2016/0066863 A1
(43) Pub. Date: Mar. 10, 2016

(54) METHOD AND APPARATUS FOR DETERMINING SpO2 OF A SUBJECT FROM AN OPTICAL MEASUREMENT

(71) Applicant: NITTO DENKO CORPORATION, Ibaraki, Osaka (JP)

(72) Inventors: Visit THAVEEPRUNGSRIPORN, Singapore (SG); Kittipong KASAMSOOK, Singapore (SG)

(73) Assignee: Nitto Denko Corporation, Osaka (JP)

(21) Appl. No.: 14/782,305

(22) PCT Filed: Apr. 5, 2013
(86) PCT No.: PCT/SG2013/000140

§ 371 Date: Oct. 2, 2015

(51) Int. Cl. A61B 5/00; A61B 5/1455

(52) U.S. Cl. CPC A61B 5/7278; A61B 5/14551; A61B 5/7246

(57) ABSTRACT

A method 100 and apparatus for determining SpO2, of a subject from an optical measurement is disclosed herein. In a described embodiment, the method 100 includes obtaining a PPG(red) signal and a PPG(IR) signal at steps 102 and 104, and pairing the PPG(red) and PPG(IR) signals at step 106 in which an amplitude of each cardiac rhythm cycle of the first signal is aligned to an amplitude of a respective cardiac rhythm cycle of the second signal to form a plurality of paired windows. At step 108, the method further includes, for each paired window, calculating values of a ratio R from the paired first and second signals and based on the calculated R values, binning the calculated R values into predetermined frequency bins at step 110. At steps 112 and 1 14, at least one of the frequency bins is selected to derive a revised ratio, Rᵣₑᵥ and at step 1 16, Sp02 is derived from the revised ratio Rᵣₑᵥ. A zoning schema for deriving SpO2 is also disclosed.

### BACKGROUND AND FIELD OF THE INVENTION

This invention relates to a method and apparatus for determining blood oxygen saturation level, SpO2, of a subject from an optical measurement.

In pulse oximetry, it is known to pass light of two different wavelengths, such as red and infrared (IR), to a part of a patient's body (such as fingertip etc) to a photodetector. The light detected at the photodetector is then analysed to obtain AC and DC components of the respective red and infrared light. Based on the AC and DC components, oxygen saturation level (SpO2) of the patient may be obtained.

Ambient light sources such as fluorescent lights, halogen lamps and sunlight radiate at frequencies similar to red and infrared light and thus, this may affect the accuracy of SpO2 obtained in this manner. As a result, noise filtering techniques have been proposed methods to eliminate the influence of such ambient light sources. For example, in subtractive noise filtering, an ambient light photoplethysmography (PPG) signal is measured and subtracted from an input signal. The ambient light PPG signal is a signal detected by the photodetector when both the red and infrared light sources (such as LEDs) are turned off.

In another example, frequency-based filtering may be used which uses complex transforms (FFT, Cepstrum etc) and signal flows to remove signal artefacts caused by both movement and the ambient light sources.

Such noise filtering techniques, however, are mathematically complex and thus, involve significant computation resource that may consume large amounts of power. As a result, such techniques are not suitable for a portable pulse oximeter with limited processing power.

### SUMMARY OF THE INVENTION

In accordance with a first aspect of the present invention, there is provided a method of determining SpO2 of a subject from an optical measurement, the method comprising:
(i) obtaining a first signal from a first light illumination;
(ii) obtaining a second signal from a second light illumination, the second light illumination having a wavelength which is different from that of the first light illumination;
(iii) pairing the first and second signals in which an amplitude of each cardiac rhythm cycle of the first signal is aligned to an amplitude of a respective cardiac rhythm cycle of the second signal to form a plurality of paired windows;
(iv) for each paired window, calculating values of a ratio R from the paired first and second signals;
(v) based on the calculated R values, binning the calculated R values into predetermined frequency bins; and
(vi) selecting at least one of the frequency bins to derive a revised ratio, Rᵣₑᵥ; and
(vii) determining SpO2 from the revised ratio Rᵣₑᵥ.

An advantage of the described embodiment is that more reliable and accurate SpO2 may be derived from the revised ratio Rᵣₑᵥ in view of the non-linear relationship between R and SpO2. Further, with the frequency bins, rules may be used to select an appropriate bin or bins and this is less complex and does not require many calculations and thus, the proposed method may be suitable for implementation on portable or mobile devices.

The predetermined frequency bins may have a fixed width. In the alternative, the predetermined frequency bins may have a variable width that is adjustable dynamically.

Preferably, two or more frequency bins are selected to derive the revised ratio. It is envisaged that selecting at least one of the frequency bins may include applying heuristic rules to the binned R values of the frequency bins. The heuristic rules may include checking the frequency bins to determine a number of maximum bins based on number of R values. If there is one maximum bin, the method may further include checking if frequency of the maximum bin exceeds a threshold, and if the threshold is exceeded, selecting the one maximum bin for deriving Rᵣₑᵥ. The one maximum bin may include a plurality of binned R values which are averaged to derive Rᵣₑᵥ.

If there are two maximum bins, the method may further include checking if the two maximum bins are directly adjacent to each other; and if the two maximum bins are directly adjacent to each other, checking if frequency of the two maximum bins exceeds a threshold, and if the threshold is exceeded, selecting the two maximum bins for deriving Rᵣₑᵥ. If the two maximum bins are not directly adjacent to each other, the method may further include checking if there is an additional bin between the two maximum bins; and if there is, selecting the additional bin and the two maximum bins for deriving Rᵣₑᵥ. If there are more than one additional bins between the two maximum bins, the method may further include rejecting the first and second signal waveforms.

If the heuristic rules determine that there are three maximum bins, and the method may further include checking if the three maximum bins are directly adjacent to each other; and if the three maximum bins are directly adjacent to each other, selecting the three maximum bins for deriving Rᵣₑᵥ. If the three maximum bins are not directly adjacent to each other, the method may further include checking if there are two maximum bins which are directly adjacent to each other; and if there are, checking if frequency of the two directly adjacent maximum bins exceeds a threshold, and if the threshold is exceeded, selecting the two directly adjacent maximum bins for deriving Rᵣₑᵥ. If there are no two maximum bins which are directly adjacent to each other, the method may include rejecting the first and second signal waveforms.

From the above checks, if the threshold is not exceeded, the method may include selecting at least one peripheral bin directly adjacent to the maximum bin or bins; and calculating if the frequency of the at least one peripheral bin and the maximum bin or bins exceeds a further threshold to determine if only the maximum bin or bins are selected or the at least one peripheral bin is selected together with the maximum bin or bins for deriving Rᵣₑᵥ.

Preferably, R is calculated based on

$$R = \frac{[ \frac{1}{{PD}_{response ( red )}} ] [ \frac{{AC}_{red}}{{DC}_{red}} ]}{[ \frac{1}{{PD}_{response ( IR )}} ] [ \frac{{AC}_{IR}}{{DC}_{IR}} ]}$$

where,
PD_{response(red)} is a response factor of the first light illumination, wherein the first light illumination is red light;
PD_{response(IR)} is a response factor of the second light illumination, wherein the second light illumination is infrared light;
AC_{red} is an AC component value of the red light;
DC_{red} is a DC component value of the red light;
AC_{IR} is an AC component value of the infrared light; and
DC_{IR} is a DC component value of the infrared light.

Preferably, SpO2 is derived from the revised ratio Rᵣₑᵥ based on:

$$\frac{( a - b \times {R}_{rev} )}{( m - n \times {R}_{rev} )}$$

where
a=ε_{Hb}(λ_{R})
b=ε_{Hb}(λ_{IR})
m=ε_{Hb}(λ_{R})−ε_{HbO}_{₂}(λ_{R})
n=ε_{Hb}(λ_{IR})−ε_{HbO}_{₂}(λ_{IR})
and where,
ε_{Hb}(λ_{R}) is an extinction coefficient of hemoglobin at Red wavelength;
ε_{Hb}(λ_{IR}) is an extinction coefficient of hemoglobin at IR wavelength;
ε_{HbO}_{₂}(λ_{R}) is an extinction coefficient of oxyhemoglobin at Red wavelength, and
ε_{Hbo}_{₂}(λ_{IR}) is extinction coefficient of oxyhemoglobin at IR wavelength.

Advantageously, the method may further comprise
(i) obtaining AC_{amb} and DC_{amb};
(ii) calculating a ratio, R_{amb-signal}, based on AC_{amb} and AC_{signal};
   where
AC_{amb} is an AC component value of an ambient signal with the first and second light illuminations switched off;
DC_{amb} is a DC component value of the ambient signal;
AC_{signal} is an AC component value of the first or second signal;
wherein, the method further comprises
(iii) providing a zoning schema divided into a plurality of reference zones with each reference zone defined by respective values of first, second and third reference variables which are associated respectively with Rᵣₑᵥ, R_{amb-signal} and DC_{amb}; each reference zone being associated with a corresponding method of computing SpO2;
(iv) comparing Rᵣₑᵥ, R_{amb-signal} and DC_{amb} with respective reference variables of the zoning schema to determine which one of the plurality of reference zones is a selected zone; and
(v) applying the method of computing SpO2 corresponding to the selected zone for determining SpO2.

Indeed, the above features may be practiced independently from the first aspect and in accordance with a second aspect of the invention, there is provided a method of determining SpO2 of a subject from an optical measurement, the method comprising:
(i) in a first interval, obtaining a first signal from a first light illumination and a second signal from second light illumination, the second light illumination having a wavelength which is different from that of the first light illumination;
(ii) calculating values of a ratio R from the first and second signals;
(iii) in a second interval, obtaining an ambient signal with the first and second light illuminations switched off;
(iv) obtaining AC_{amb} and DC_{amb}
(v) calculating a ratio, R_{amb-signal}, based on AC_{amb} and AC_{signal};
   where
AC_{amb} is an AC component value of the ambient signal;
DC_{amb} is a DC component value of the ambient signal;
AC_{signal} is an AC component value of the first or second signal;
wherein, the method further comprises
(vi) providing a zoning schema divided into a plurality of reference zones with each reference zone defined by respective values of first, second and third reference variables which are associated respectively with R, R_{amb-signal} and DC_{amb}; each reference zone being associated with a corresponding method of computing SpO2;
(vii) comparing R, R_{amb-signal} and DC_{amb} with respective reference variables of the zoning schema to determine which one of the plurality of reference zones is a selected zone; and
(viii) applying the method of computing SpO2 corresponding to the selected zone for determining SpO2.

With the zoning schema as proposed in the described embodiment, a fine-grained multi-dimensional R—SpO2 relationship characterization may be achieved, resulting in a more accurate prediction or determination of SpO2.

Preferably, the method may comprise obtaining a revised ratio, Rᵣₑᵥ based on R and using Rᵣₑᵥ in place of R at step (vii) of the second aspect. The method may further comprise pairing the first and second signals in which an amplitude of each cardiac rhythm cycle of the first signal is aligned to an amplitude of a respective cardiac rhythm cycle of the second signal to form a plurality of paired windows. Further, the method may comprise for each paired window, calculating values of the ratio R from the paired first and second signals; based on the calculated R values, binning the calculated R values into predetermined frequency bins; and selecting at least one of the frequency bins to derive the revised ratio, Rᵣₑᵥ. Advantageously, the predetermined frequency bins may have a variable width that is adjustable dynamically.

According to a third aspect, there is provided a method of determining SpO2 of a subject from an optical measurement, the method comprising:
(i) in a first interval, obtaining a first signal from a first light illumination and obtaining a second signal from a second light illumination, the second light illumination having a wavelength which is different from that of the first light illumination;
(ii) pairing the first and second signals in which an amplitude of each cardiac rhythm cycle of the first signal is aligned to an amplitude of a respective cardiac rhythm cycle of the second signal to form a plurality of paired windows;
(iii) for each paired window, calculating values of a ratio R from the paired first and second signals;
(iv) based on the calculated R values, binning the calculated R values into predetermined frequency bins; and
(v) selecting at least one of the frequency bins to derive a revised ratio, Rᵣₑᵥ;
(vi) in a second interval, obtaining an ambient signal with the first and second light illuminations switched off;
(vii) obtaining AC_{amb} and DC_{amb};
(viii) calculating a ratio, R_{amb-signal}, based on AC_{amb} and AC_{signal};
   where
AC_{amb} is an AC component value of the ambient signal;
DC_{amb} is a DC component value of the ambient signal;
AC_{signal} is an AC component value of the first or second signal;
wherein, the method further comprises
(viiii) providing a zoning schema divided into a plurality of reference zones with each reference zone defined by respective values of first, second and third reference variables which are associated respectively with Rᵣₑᵥ, R_{amb-signal} and DC_{amb}; each reference zone being associated with a corresponding method of computing SpO2;
(x) comparing Rᵣₑᵥ, R_{amb-signal} and DC_{amb} with respective reference variables of the zoning schema to determine which one of the plurality of reference zones is a selected zone; and
(xi) applying the method of computing SpO2 corresponding to the selected zone for determining SpO2.

According to a fourth aspect, there is provided an optical measurement apparatus for determining SpO2 of a subject, the apparatus comprising
a photodetector configured to obtain a first signal from a first light illumination and a second signal from a second light illumination, the second light illumination having a wavelength which is different from that of the first light illumination; and
a processor configured to
(i) pair the first and second signals in which an amplitude of each cardiac rhythm cycle of the first signal is aligned to an amplitude of a respective cardiac rhythm cycle of the second signal to form a plurality of paired windows;
(ii) for each paired window, calculate values of a ratio R from the paired first and second signals;
(iii) based on the calculated R values, bin the calculated R values into predetermined frequency bins; and
(iv) select at least one of the frequency bins to derive a revised ratio, Rᵣₑᵥ; and
(v) determine SpO2 from the revised ratio Rᵣₑᵥ.

According to a fifth aspect, there is provided an optical measurement apparatus for determining SpO2 of a subject, the apparatus comprising:
(a) a photodetector configured to
(i) in a first interval, obtain a first signal from a first light illumination and a second signal from second light illumination, the second light illumination having a wavelength which is different from that of the first light illumination;
(ii) in a second interval, obtain an ambient signal with the first and second light illuminations switched off;
(b) a processor configured to:
(iii) calculate values of a ratio R from the first and second signals;
(iv) obtain AC_{amb} and DC_{amb};
(v) calculate a ratio, R_{amb-signal}, based on AC_{amb} and AC_{signal};
   where
AC_{amb} is an AC component value of the ambient signal;
DC_{amb} is a DC component value of the ambient signal;
AC_{signal} is an AC component value of the first or second signal; and
(c) a zoning schema divided into a plurality of reference zones with each reference zone defined by respective values of first, second and third reference variables which are associated respectively with R, R_{amb-signal} and DC_{amb}; each reference zone being associated with a corresponding method of computing SpO2;
wherein the processor is further configured to:
(vi) compare R, R_{amb-signal} and DC_{amb} with respective reference variables of the zoning schema to determine which one of the plurality of reference zones is a selected zone; and
(vii) apply the method of computing SpO2 corresponding to the selected zone for determining SpO2.

It should be appreciated that the described embodiment may be used for human and/or animal subjects.

It should be apparent that features applicable for one aspect may also be applicable for the other aspects.

### BRIEF DESCRIPTION OF THE DRAWINGS

An example of the invention will now be described with reference to the accompanying drawings, in which:

FIG. 1 is shows steps of a method of determining SpO2 of a subject from an optical measurement according to a preferred embodiment of the invention;

FIG. 2 is a schematic block diagram of an optical measurement device for performing the optical measurement of FIG. 1 together with a telecommunications device;

FIG. 3 shows paired PPG signal waveforms obtained at pairing of PPG signal step of FIG. 1;

FIG. 4 is a graph showing frequency distribution of Ratio-of-Ratios across a number of frequency bins as obtained at binning step of FIG. 1;

FIGS. 5 to 7 are flowcharts showing steps for heuristic rules which are used for determining which bin to select in FIG. 1; and

FIG. 8 shows a zoning schema which may be used for determining SpO2 level in FIG. 1.

### DETAILED DESCRIPTION OF PREFERRED EMBODIMENT

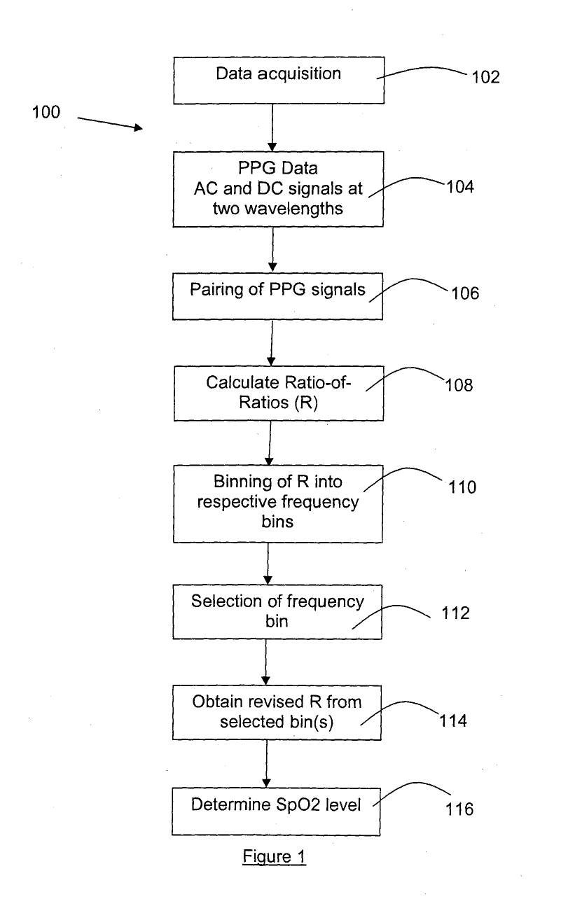

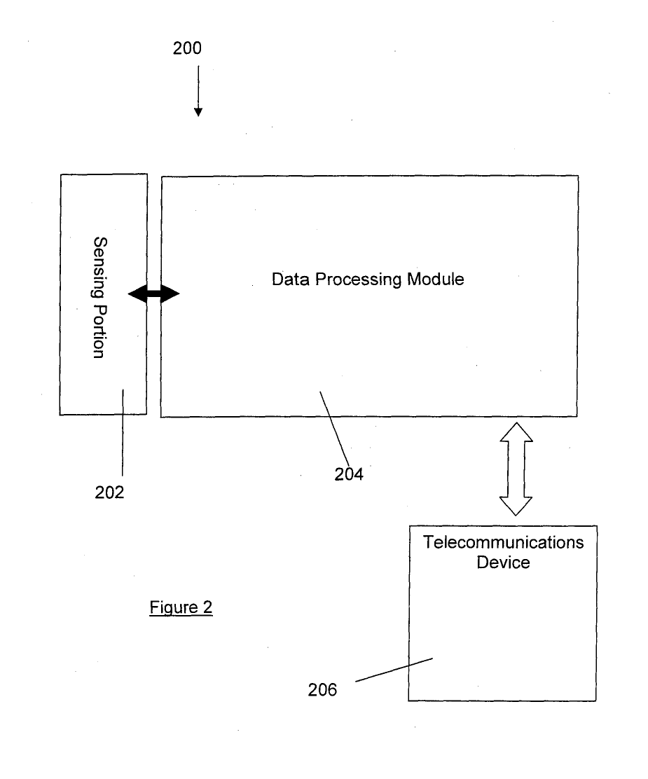

FIG. 1 shows a method 100 of determining SpO2 of a subject from an optical measurement according to a preferred embodiment of the invention. The method 100 includes data acquisition at step 102, in which an optical measurement device is used to obtain data from a subject, such as a patient. An example of an optical measurement device is one which is described in PCT/SG20012/000006, the content of which is incorporated herein by reference. FIG. 2 shows a schematic block diagram of such an optical measuring device 200 which includes a sensing portion 202 communicatively coupled to a data processing module 204. The data processing module 204 includes a microprocessor and may then be coupled to a telecommunications device 206 such as a mobile phone of various brands as described in PCT/SG20012/000006.

The optical measurement device 200 is configured to process PPG signals and for the sake of simplicity, it would be referred to as a PPG device 200 and the sensing portion 202 includes two light illuminations in the form of emitting diodes for emitting light of two different or distinct wavelengths onto the patient's fingertip (although other parts of the patience's body may be used). In this embodiment, the two emitting diodes emit red and infrared (IR) light respectively with corresponding wavelengths of 660 nm and 940 nm. The sensing portion 202 further includes a photodiode functioning as a photodetector for receiving the data at step 104 which is in the form of PPG signals reflected or transmitted off the skin of the fingertip.

Specifically, the data acquisition step 102 is performed over two intervals, intervals (A) and (B). In interval (A), both emitting diodes are turned on for a sufficient time period (either one at a time or both together) to obtain a PPG(red) signal and a PPG(infrared) signal which correspond respectively to the red and infrared red signals reflected off the skin of the fingertip and detected at the photodiode.

For ease of reference, components associated with the red light would include the term “red” between parentheses and likewise for components associated with IR light would include the term (IR) between parentheses.

The PPG(red) signal includes a plurality of cardiac rhythm cycles or pulses of the patient as detected by the photodiode and the PPG(red) signal may generally be divided into two components: an AC(red) component as a result of absorption of the red light in pulsatile arterial blood; and a DC(red) component as a result of absorption of the red light by non-pulsatile arterial blood such as venous blood and capillary blood. Further, the PPG(red) signal would also include an ambient PPG signal due to ambient light sources near the PPG device, particularly if the PPG device does not have a cover for the fingertip.

The PPG(IR) signal may also be broadly divided similar to the PPG(red) signal and this is represented by an AC(IR) component and a DC(IR) component similar to the PPG(red) signal.

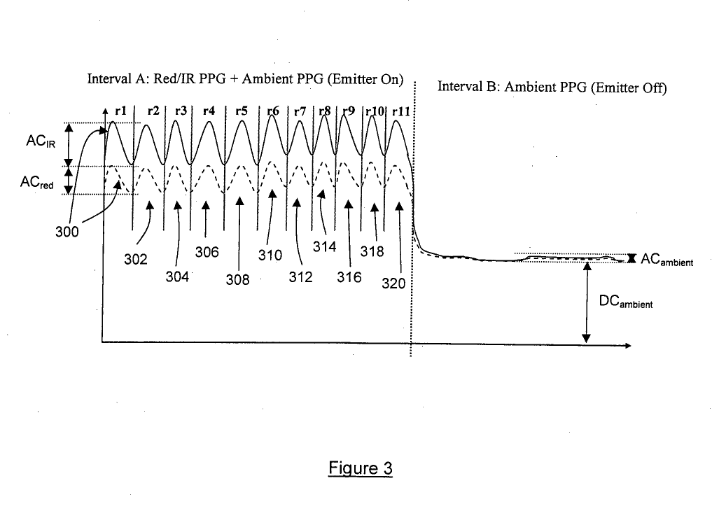

At step 106, the data processing module 204 pairs the PPG(red) and PPG(IR) signals based on amplitude of the PPG(red) and PPG(IR) signals to form respective pairs of PPG(red) and PPG(IR) cardiac cycles 300,302,304 . . . 320 as shown in FIG. 3. Accordingly, the data processing module 204 includes a peak detector to perform the pairing. In this embodiment, maximum amplitudes of the PPG(red) and PPG(IR) signals are used, although it is possible that minimum amplitudes may be used for the pairing.

In this embodiment, there are eleven pairs of the PPG(red) and PPG(IR) cardiac cycles and the paired PPG(red) and PPG(IR) cardiac cycles 300,302,304 . . . 320 are next divided into a plurality of sampling windows r1,r2,r3 . . . r11 with each sampling window having one pair of PPG(red) and PPG(IR) cardiac cycle. For each sampling window r1,r2,r3 . . . r11, the data processing module calculates a ratio R, commonly known as Ratio of Ratios, at step 108 (see FIG. 1) using equation (1) below, which is derived from Beer-Lambert Law:

$$R = \frac{[ \frac{1}{{PD}_{{response }_{red}}} ] [ \frac{{AC}_{red}}{{DC}_{red}} ]}{[ \frac{1}{{PD}_{{response }_{IR}}} ] [ \frac{{AC}_{IR}}{{DC}_{IR}} ]} \qquad Equation 1$$

where,
AC_{red} is a value of the AC(red) component of the PPG(red) signal;
DC_{red} is a value of the DC(red) component of the PPG(red) signal;
AC_{IR} is a value of the AC(IR) component of the PPG(IR) signal;
DC_{IR} is a value of the DC(IR) component of the PPG(IR) signal;
PD_{response(red)} is a PD factor of the photodiode (PD) for the PPG(red) signal; and
PD_{response(IR)} is a PD factor of the photodiode (PD) for the PPG(IR) signal.

In this way, the ratio R obtained is normalized to the response of the photodiode and thus, provides a more accurate value of R. As an example, for a photodiode model TSL13D, the PD_{response(red)} is 0.87 and the PD_{response(IR)} is 0.63. It should be appreciated that the ratio R may not be normalized to the response of the photodiode and if this is the case, then R would be derived from the AC_{red}, DC_{red}, AC_{IR} and DC_{IR} values as shown above (i.e. without the first set of square brackets in equation (1)).

Based on equation (1), the ratio R for each sampling window is derived as shown in Table 1 below:

TABLE 1

| | Sample Window | | | | | | | | | | |
|---|---|---|---|---|---|---|---|---|---|---|---|
| | r1 | r2 | r3 | r4 | r5 | r6 | r7 | r8 | r9 | r10 | r11 |
| R values | 0.12 | 0.51 | 0.55 | 0.46 | 0.59 | 0.78 | 0.56 | 0.68 | 0.65 | 0.27 | 0.98 |

Based on Table 1, the values of R for each sampling window r1, r2, r3 . . . r11 are next binned at step 110 to respective frequency bins based on a bin width or interval according to their corresponding values of R, as shown in Table 2:

TABLE 2

| | | Numerical | Sample | Frequency | |
|---|---|---|---|---|---|
| Bin | Width | Range | Windows | (counts) | % |
| a | 0.1 | 0.1 <= r < 0.2 | r1 | 1 | 9.09% |
| b | 0.1 | 0.2 <= r < 0.3 | r10 | 1 | 9.09% |
| c | 0.1 | 0.3 <= r < 0.4 | None | 0 | 0% |
| d | 0.1 | 0.4 <= r < 0.5 | r4 | 1 | 9.09% |
| e | 0.1 | 0.5 <= r < 0.6 | r3, r5, r2, r7 | 4 | 36.36% |
| f | 0.1 | 0.6 <= r < 0.7 | r8, r9 | 2 | 18.18% |
| g | 0.1 | 0.7 <= r < 0.8 | r6 | 1 | 9.09% |
| h | 0.1 | 0.8 <= r < 0.9 | None | 0 | 0% |
| i | 0.1 | 0.9 <= r < 1.0 | r11 | 1 | 9.09% |
| | | | Total | 11 | |

In this embodiment, there are nine bins, Bin a,b,c . . . i with a bin width of 0.1 which determines the spread of the Numerical Range in Table 2. The bin width of 0.1 is derived from experimental results. Each bin a,b,c . . . i represents a frequency of occurrence of a particular numerical interval of the ratio R.

It should be appreciated Table 2 illustrates the binning of the sample windows r1,r2 . . . r11 but indeed, the binning is performed based on the values of R and the references for the sample windows are merely used as another form of representing the values of R.

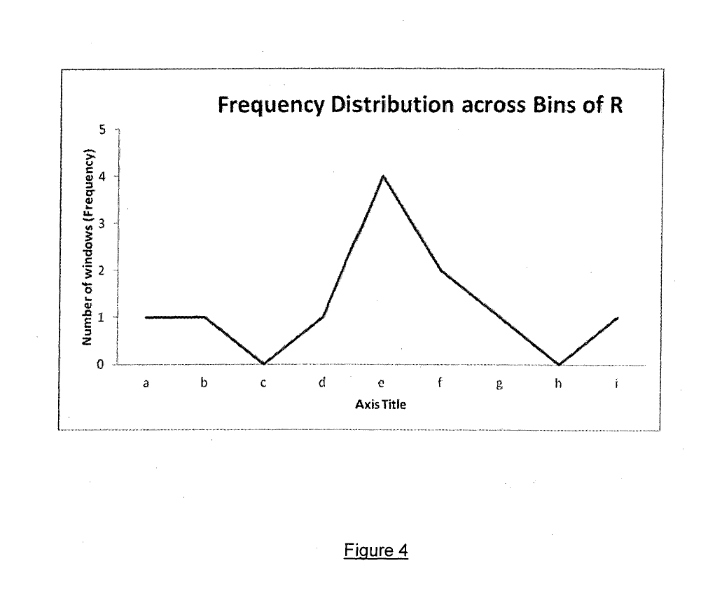

FIG. 4 is a graphical representation of the frequency distribution of the ratio R across the various bins a,b,c . . . j, and this is another example of how the data processing module 204 may present the binning (for example via a display).

After binning of the values of the ratio R, at step 112, the data processing module 204 applies a set of heuristic rules to select a bin or bins to obtain a revised ratio, Rᵣₑᵥ, in the following order:
1. Rule 1;
2. Rule 2;
3. Rule 3; and
4. Rule 4.

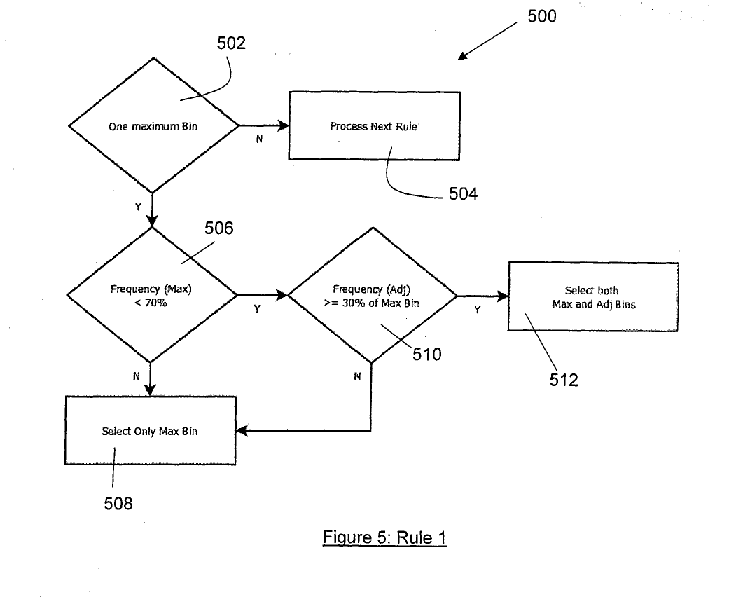

FIG. 5 is a flowchart 500 showing steps for Rule 1 executed by the data processing module 204. At step 502, the data processing module 204 checks if there is a single maximum bin which means the bin with the most number of values of the ratio R. If there is no one single maximum bin, step 504 is executed and the data processing module 204 goes to the next rule which is Rule 2.

If there is one single maximum bin, the data processing module 204 goes to step 506 to check if a maximum frequency of the bin is less than 70%. The maximum frequency in this context means maximum number of counts in the single maximum bin (for example, in table 2, bin e has a maximum frequency/count of 4) expressed as a % of the total counts. For example, in table 2, bin e has a maximum frequency/count of 4 and when this is expressed as a %, this is 4/11 which is 36.36%. If the maximum frequency is not less than 70% (i.e. it is more than 70%), step 508 is executed by the data processing module 204 which selects the maximum bin.

On the other hand, if the maximum frequency at step 506 is found to be less than 70%, step 510 is executed which checks if frequencies of the directly adjacent bins (or bin, depending on where the maximum bin is located in the distribution) i.e. Frequency(adj) is more than or equal to 30% of the maximum bin. If no, then step 508 is again executed to select the maximum bin. If the Frequency(adj) is more than or equal to 30% of the maximum bin, step 512 is executed which selects both the maximum bin and adjacent bins (i.e. bins directly adjacent to the maximum bin). It should be appreciated that there may be just one adjacent bin when, for example, the maximum bin is at an extreme end of the distribution.

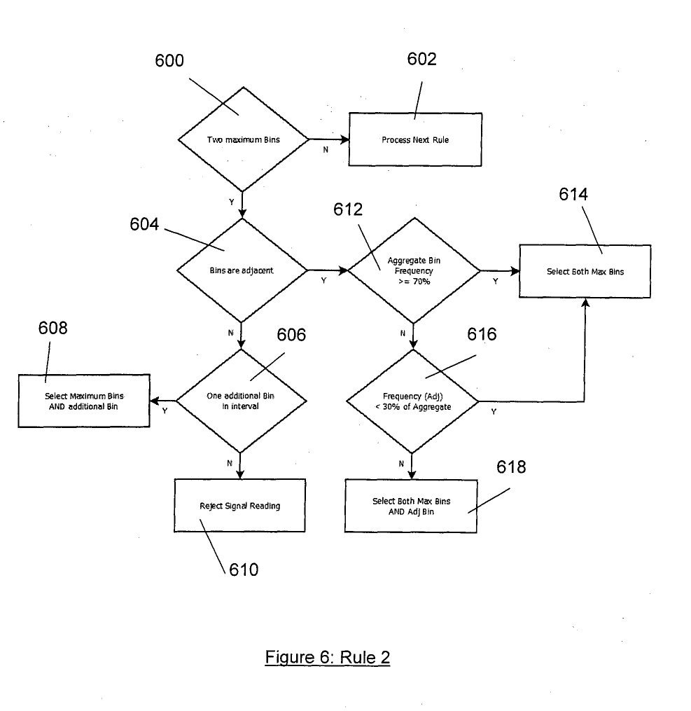

FIG. 6 is a flowchart showing Rule 2 which is executed after step 504 of FIG. 5. At step 600, the data processing module 204 checks the binning distribution of Table 2 if there are two maximum bins. If there is none, step 602 is executed and the data processing module 204 processes the next rule, which is Rule 3.

If two maximum bins are found, the data processing module 204 executes step 604 to determine if the two maximum bins are directly adjacent to each other. If no, step 606 is executed to determine if the two maximum bins have one additional bin sandwiched between the two maximum bins. If yes, step 608 prompts the data processing module 204 to select the two maximum bins and the one additional bin. If the two maximum bins have more than one additional bin sandwiched in-between, the data processing module 204 executes step 610 to reject the PPG(red) and PPG(IR) signals and perhaps, displays an error message to the patient for readings to be taken again.

At step 604, if the two maximum bins are directly adjacent to each other, the data processing module 204 executes step 612 to check if an aggregate bin frequency is more than or equal to 70%. By aggregate bin frequency, this means that the bin frequencies of both bins are added together (for example, the aggregate frequencies of bins a and b of Table 2 is 18.18%). If yes, step 614 requires the data processing module 204 to select both the two maximum bins which ends Rule 2.

On the other hand, if the aggregate bin frequency is not more than or equal to 70%, at step 616, the frequency of the adjacent bin(s) is checked if the frequencies of these adjacent bin(s) as a function of the aggregate bin frequencies of the two maximum bins is less than 30%. If yes, step 614 is executed again which select both the maximum bins. If no, step 618 selects both the two maximum bins and the adjacent bin(s) directly next to the two maximum bins which has an aggregate of more than 30%.

Table 3 is a table showing another example of binning of values of R, which are different from those illustrated in Table 2, to illustrate how step 618 is performed.

TABLE 3

| | | Numerical | Sample | Frequency | | |
|---|---|---|---|---|---|---|
| Bin | Width | Range | Windows | (counts) | % | |
| a | 0.1 | 0.1 <= r < 0.2 | None | 0 | 0% | |
| b | 0.1 | 0.2 <= r < 0.3 | None | 0 | 0% | |
| c | 0.1 | 0.3 <= r < 0.4 | r7 | 1 | 9.09% | |
| | | | | | (1/11) | |
| d | 0.1 | 0.4 <= r < 0.5 | r4, r10 | 2 | 18.18% | 33.33% of |
| | | | | | (2/11) | max. bin (2/6) |
| e | 0.1 | 0.5 <= r < 0.6 | r2, r3, r5 | 3 | 27.27% | 54.54% (6/11) - |
| | | | | | (3/11) | Combined bin |
| f | 0.1 | 0.6 <= r < 0.7 | r1, r6, r8 | 3 | 27.27% | |
| | | | | | (3/11) | |
| g | 0.1 | 0.7 <= r < 0.8 | r11 | 1 | 9.09% | 16.67% of |
| | | | | | (1/11) | max. bin (1/6) |
| h | 0.1 | 0.8 <= r < 0.9 | r9 | 1 | 9.09% | |
| | | | | | (1/11) | |
| i | 0.1 | 0.9 <= r < 1.0 | None | 0 | 0% | |
| | | | Total | 11 | | |

In Table 3, it can be appreciated that the two maximum bins e and f are located adjacent to each other and thus, Rule 2 of FIG. 6 would calculate the aggregate bin frequency at step 612 as explained above. In this respect, the aggregate frequency of the combined bin is 54.54% of the total counts of 11. Since the aggregate frequency is less than 70%, Rule 2 next branches to step 616 to calculate the frequencies of the adjacent bins. The bins adjacent to the two maximum bins e and f are bins d and g and the aggregate frequency of bin d, based on the aggregate count of the two maximum bins (which is 3+3=6) is thus 33.33%. The frequency of the other adjacent bin (bin g) is 16.67%. Thus, the selected bins for this R calculation at step 618 are bins d, e and f (since the % of bin d is more than 30%).

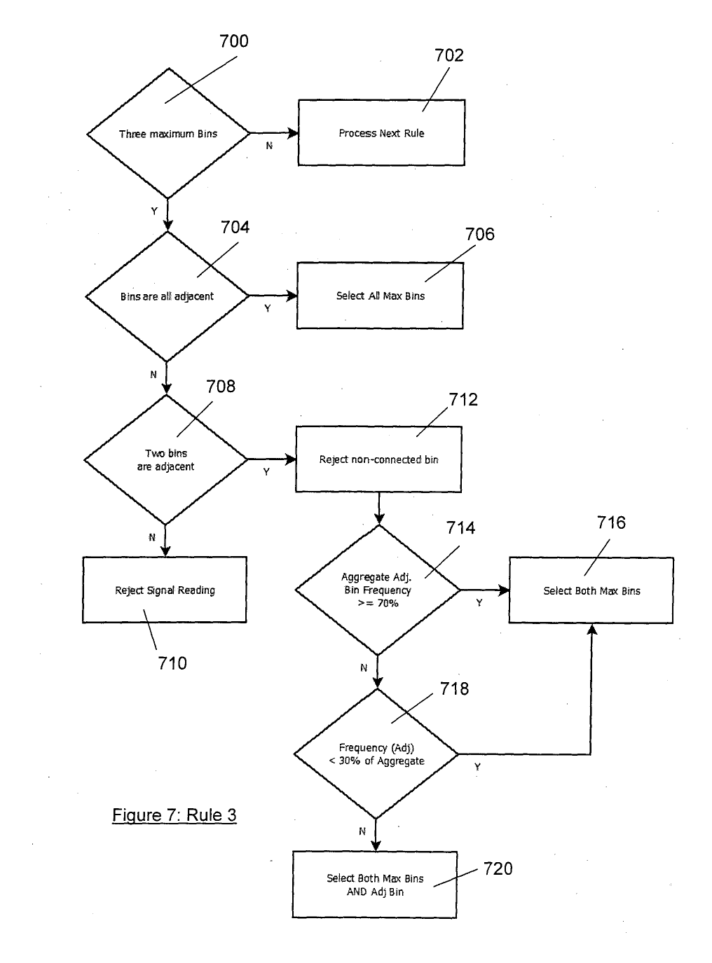

At step 602, to process the next rule, the data processing module 204 goes to Rule 3 which is illustrated as a flowchart in FIG. 7.

The data processing module 204 checks if there are three maximum bins at step 700. If there is none, step 702 is executed and the next rule (i.e. Rule 4) is executed. If there are three maximum bins, step 704 is executed to determine if all the three maximum bins are directly adjacent or next to each other. If yes, all the three maximum bins are selected at step 706.

If the three maximum bins are not directly adjacent to each other, step 708 checks if two of the bins are directly adjacent to each other. If no, at step 710, the PPG(red) and PPG(IR) signals are rejected similar to the situation at step 610 of FIG. 6 (Rule 2). If there are two directly adjacent maximum bins out of the three maximum bins, at step 712, the “non-connected” maximum bin is rejected and for the two “connected” or directly adjacent maximum bins, step 714 aggregates the bin frequencies of the two directly adjacent maximum bins. If the aggregate of the bin frequencies exceeds or is equal to 70%, both of the directly adjacent maximum bins are selected at step 716. On the other hand, if the aggregate of the bin frequencies is not more than or equal to 70%, step 718 is performed to check if the frequency of the bin(s) directly adjacent to the two maximum bins is less than 30% of the aggregate (of the bin frequencies of the two maximum bins). If yes, then step 716 is performed again to select just the two maximum bins. On the other hand, if no, then the two maximum bins and the directly adjacent bin(s) are selected at step 720.

It should be appreciated that in the above rules, there may be one bin or two bins directly adjacent to the maximum bin(s) selected. For example, if the maximum bin(s) is located at the extreme end of the distribution, there would be one bin directly adjacent to it, whereas if the maximum bin(s) is located somewhere near the middle of the distribution, there would be two adjacent bins.

At step 702, the data processing module 204 proceeds to the next rule which is Rule 4 which rejects the PPG(red) and PPG(IR) signals since the data obtained did not meet any of Rules 1-3. No computation is performed and an error message may be displayed similar to step 610 of FIG. 6 (Rule2).

Based on Rules 1-4 as explained above, and with reference to Table 2, in this embodiment, Rule 1 applies since there is a single maximum bin in bin e in the numerical range 0.5 to 0.6. Step 506 of Rule 1 is thus executed and in this respect, the maximum frequency of bin e is 36.36% of the total count. Since this is less than 70%, Rule 1 goes to step 510 which checks the frequencies of the adjacent bins i.e. bins d and f. For bin d, there is only one count, and this is 25% of the maximum frequency (i.e. 1 count out of 4 counts of the maximum bin e). For bin f, there are two counts (r8 and r9) and this is 50% of the maximum frequency (i.e. 2 out of 4 counts of the maximum bin e), which is greater than 30%. Thus, Rule 1 goes to step 512 to select bins e and f as the selected bins. Rrev is calculated from an average value of r2,r3,r5,r7,r8 and r9 which is 0.59.

With the Rᵣₑᵥ obtained, step 116 determines the SpO2 level from the Rᵣₑᵥ based on equation (2) below:

$$SpO 2 = \frac{( a - b \times {R}_{rev} )}{( m - n \times {R}_{rev} )} \qquad Equation 2$$

where,
a,b,m and n are empirical coefficients (of Hb (Hemoglobin) and HbO₂ (oxyhemoglobin) derived from curve fitting to experimental results.

To elaborate, Equation 2 is a simplified form of an extinction coefficient equation below:

$${SPO}_{2} = \frac{{ɛ}_{Hb} ( {λ}_{R} ) - [ {ɛ}_{Hb} ( {λ}_{IR} ) \times R ]}{\begin{matrix}{ɛ}_{Hb} ( {λ}_{R} ) - {ɛ}_{{HbO}_{2}} ( {λ}_{R} ) + \\ \{ [ {ɛ}_{{HbO}_{2}} ( {λ}_{IR} ) - {ɛ}_{Hb} ( {λ}_{IR} ) ] \times R \}\end{matrix}} \qquad Equation 3$$

where,
a=ε_{Hb}(λ_{R})
b=ε_{Hb}(λ_{IR})
m=ε_{Hb}(λ_{R})−ε_{HbO}_{₂}(λ_{R})
n=ε_{Hb}(λ_{IR})−ε_{HbO}_{₂}(λ_{IR})
and where,
ε_{Hb}(λ_{R}) is an extinction coefficient of hemoglobin at Red wavelength (660 nm in this embodiment);
ε_{Hb}(λ_{IR}) is an extinction coefficient of hemoglobin at IR wavelength (940 nm),
ε_{HbO}_{₂}(Δ_{R}) is an extinction coefficient of oxyhemoglobin at Red, and
ε_{HbO}_{₂}(λ_{IR}) is extinction coefficient of oxyhemoglobin at IR, respectively.

All the coefficients (a, b, m, and n) are derived from curve fitting to the experimental result. For example, if Rᵣₑᵥ=0.59 is substituted into Equation 3, SPO2 is calculated to be about 96.86%→97% (round up).

SpO2 is then displayed to the patient or transmitted to be stored or for review by a medical professional (for example, via the telecommunications device coupled to the PPG device).

By obtaining the revised ratio Rᵣₑᵥ, the method is able to derive a more accurate reading for SpO2. Such a method is also requires less computational power and thus suitable to be implemented for portable devices. With the method, identification and classification of outlier readings via heuristic examination of a variable distribution (i.e. R) may be achieved which enables a more reliable numerical average of the desired variable to be obtained.

It should be appreciated that SpO2 may be derived from equation 2 but it is also possible that refinements be made to improve the accuracy of SpO2 by taking into account an ambient signal caused by ambient sources around the PPG device. Referring to FIGS. 1 and 2, at step 102, in addition to interval A after obtaining the PPG(red) and PPG(IR) signals, during interval (B), the two illuminations are switched off and the PPG device 200 is thus configured to detect the ambient signal and as shown in FIG. 1, the ambient signal may also be broadly divided into AC_{amb} and DC_{amb} which are AC component and DC component values of the ambient signal.

Over intervals (A) and (B), the values obtained are:
i. AC_{amb};
ii. AC_{red};
iii. AC_{IR};
iv. DC_{amb};
v. DC_{red}; and
vi. DC_{IR}.

For the purposes of obtaining SpO2, a ratio AC_{amb}/AC_{red} is calculated and this together with Rᵣₑᵥ and DC_{amb}, which are measured from the patient, are used for computing SpO2 as explained below.

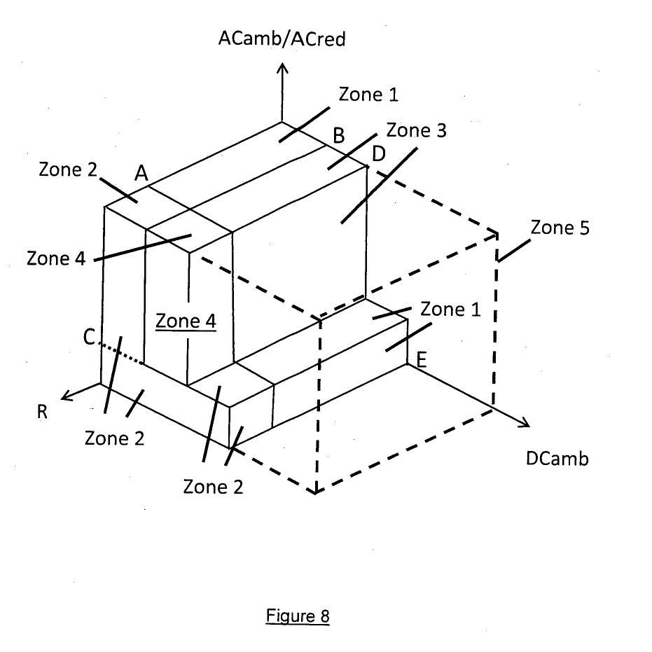

FIG. 8 illustrates a zoning schema for computing SpO2. The zoning schema includes a plurality of reference zones with each zone defined by respective values of three variables, which are:
First variable: reference ratio, R_{ref} which corresponds to Rᵣₑᵥ or the ratio R, as the case may be;
Second variable: reference AC_{amb}/AC_{red} which corresponds to the ratio AC_{amb}/AC_{red}
Third variable: reference DC_{amb} which corresponds to DC_{amb}.

As it can be appreciated from FIG. 8, each of these reference variables R_{ref}, DC_{amb} and AC_{amb}/AC_{red} correspond to X,Y and Z axes when the zoning schema is represented three-dimensionally. The values of these reference variables are determined by experimentation or empirically and the zones are demarcated or delineated by points A,B,C,D and E located along one of the X,Y,Z axes and in this embodiment, there are five distinctive zones—Zones 1-5. Each point A,B,C,D and E is defined by a value of one of the three reference variables and exact values of points A,B,C,D and E are also determined by experimentation or empirically and preprogrammed into the PPG device 200 (perhaps in the factory).

Again, the zoning schema with the associated methods of computation are preprogrammed into the PPG device and when the measured values, the ratio AC_{amb}/AC_{red} Rᵣₑᵥ and DC_{amb}, are obtained, these measured values are compared against the zoning schema to determined which one of the five zones these measured values fall into.

With the proposed zoning schema, this is particularly useful to enhance SpO2 calculation since the relationship between R (or Rᵣₑᵥ) and SpO2 is non-linear and is sensitive to input variables including noise. In particular, a fine-grained, multi-dimensional R-SpO2 relationship characterization may be achieved which takes into account the ambient signal by the proposed method.

It should be appreciated that the zoning schema may not be only used for the method of FIG. 1 (i.e. to obtain a revised R) but instead, the zoning schema may also be directly used with the initial ratio R, or applied to other methods of noise reducing methods to derive R and in turn the zoning schema is used to provide an improved SpO2 reading.

It is possible that the points A,B,C,D and E may be configured to change dynamically during the operation of the PPG device to further enhance the capability of this method In the described embodiment, each zone is assigned a corresponding method of computation to derive a SpO2 value from the measured PPG data obtained from the patient. It can be appreciated that the corresponding method may be shared between different zones (for example, Zones 2 and 4 share the same method) or may be completely unique to respective zones, depending on the application and the type of optical measurement device. The method of computation may involve a first formula and associated coefficients describing a relationship between R and SpO2 that may be derived empirically from methods of (spline) curve fitting or regression (for example, Equation (3) associated with Zone 1). The method of computation may also involve initial compensation of the measured input value (for example, compensation of the revised R, Rᵣₑᵥ) by a first formula and associated coefficients before being processed by a second formula and associated coefficients associated with the compensation (e.g. R_{comp}) to derive SpO2 (for example, in the case of Zone 3). The formulae and coefficients may also be derived empirically through the use of curve fitting or regression. Furthermore, the method of computation may involve discarding the measurements and outputting a null value to indicate presence of a particularly poor measurement (for example, when the measured data fall within Zone 5). The formulae associated with each method may include input dimensions over and above the input variable R. The zoning schema with the plurality of zones may be preprogrammed into the device or stored in a memory of the device.

The described embodiment should not be construed as limitative. For example, in the described embodiment, the optical measurement device is described having the sensing portion and the data processing module which is coupled to a mobile telecommunications device such as a mobile phone. However, it is possible that the method may also be used for other optical measurement devices such as a pulse oximeter. Also, the method may also be applicable for reflectance and transmissive pulse oximetry.

The disclosed method is particularly useful for pulse oximeters or optical measurement devices which do not have a “cover” (for example, cover for the patients' fingertip) and thus, the measurement is more exposed to ambient light sources.

The described embodiment uses PPG data as an example but the proposed method may also be used for ECG data or other types of optical measurements. Other types of light may be used, not just red and IR and certainly, other suitable wavelengths may be selected, not just 660 nm and 940 nm.

As shown in Table 2, the bin width is 0.1 and the bin width may be obtained by experimental results or arbitrarily defined and may cover any relevant range of the ratio R based on application or the PPG device. It should be appreciate also that the bin width may be dynamically adjusted while the PPG device is in operation.

The heuristic rules used to determine the selected bin may constitute a single rule or multiple rules constituting a given set. These rules may be preprogrammed into the device on manufacture (or at the factory), may be stored in a memory and may include adjustable parameters that may be modified during device operation. These rules may be used individually or chained in tandem to device which grouping or sets of groupings to use to obtain a revised R (for example, through averaging). To obtain the revised R, simple averaging may be used or in the alternative, weighed averaging may also be used.

Having now fully described the invention, it should be apparent to one of ordinary skill in the art that many modifications can be made hereto without departing from the scope as claimed.

1. A method of determining SpO2 of a subject from an optical measurement, the method comprising:
(i) obtaining a first signal from a first light illumination;
(ii) obtaining a second signal from a second light illumination, the second light illumination having a wavelength which is different from that of the first light illumination;
(iii) pairing the first and second signals in which an amplitude of each cardiac rhythm cycle of the first signal is aligned to an amplitude of a respective cardiac rhythm cycle of the second signal to form a plurality of paired windows;
(iv) for each paired window, calculating values of a ratio R from the paired first and second signals;
(v) based on the calculated R values, binning the calculated R values into predetermined frequency bins; and
(vi) selecting at least one of the frequency bins to derive a revised ratio, Rᵣₑᵥ; and
(vii) determining SpO2 from the revised ratio Rᵣₑᵥ; wherein
the method further comprises
(viii) obtaining AC_{amb} and DC_{amb};
(viiii) calculating a ratio, R_{amb-signal}, based on AC_{amb} and AC_{signal};
(x) providing a zoning schema divided into a plurality of reference zones with each reference zone defined by respective values of first, second and third reference variables which are associated respectively with Rᵣₑᵥ, R_{amb-signal} and DC_{amb}; each reference zone being associated with a corresponding method of computing SpO2;
(xi) comparing Rᵣₑᵥ, R_{amb-signal} and DC_{amb} with respective reference variables of the zoning schema to determine which one of the plurality of reference zones is a selected zone; and
(xii) applying the method of computing SpO2 corresponding to the selected zone for determining SpO2;
where
AC_{amb} is an AC component value of an ambient signal with the first and second light illuminations switched off;
DC_{amb} is a DC component value of the ambient signal; and
AC_{signal} is an AC component value of the first or second signal.

2-4. (canceled)

5. A method according to claim 1, wherein selecting at least one of the frequency bins includes applying heuristic rules to the binned R values of the frequency bins.

6. A method according to claim 5, wherein the heuristic rules includes checking the frequency bins to determine a number of maximum bins based on number of R values.

7. A method according to claim 6, wherein there is one maximum bin, and the method further includes checking if frequency of the maximum bin exceeds a threshold, and if the threshold is exceeded, selecting the one maximum bin for deriving Rᵣₑᵥ.

8. A method according to claim 7, wherein the one maximum bin includes a plurality of binned R values which are averaged to derive Rᵣₑᵥ.

9. A method according to claim 6, wherein there are two maximum bins, and the method further includes
checking if the two maximum bins are directly adjacent to each other; and
if the two maximum bins are directly adjacent to each other, checking if frequency of the two maximum bins exceeds a threshold, and if the threshold is exceeded,
selecting the two maximum bins for deriving Rᵣₑᵥ.

10. A method according to claim 9, wherein if the two maximum bins are not directly adjacent to each other, the method further includes
checking if there is an additional bin between the two maximum bins; and
if there is, selecting the additional bin and the two maximum bins for deriving Rᵣₑᵥ.

11. A method according to claim 10, wherein if there are more than one additional bins between the two maximum bins, the method further includes rejecting the first and second signal waveforms.

12. A method according to claim 6, wherein there are three maximum bins, and the method further includes
checking if the three maximum bins are directly adjacent to each other; and
if the three maximum bins are directly adjacent to each other, selecting the three maximum bins for deriving Rᵣₑᵥ.

13. A method according to claim 12, wherein if the three maximum bins are not directly adjacent to each other, the method further includes
checking if there are two maximum bins which are directly adjacent to each other; and
if there are, checking if frequency of the two directly adjacent maximum bins exceeds a threshold, and
if the threshold is exceeded, selecting the two directly adjacent maximum bins for deriving Rᵣₑᵥ.

14. A method according to claim 13, wherein if there are no two maximum bins which are directly adjacent to each other, the method includes rejecting the first and second signal waveforms.

15. A method according to claim 7, wherein if the threshold is not exceeded, the method includes
selecting at least one peripheral bin directly adjacent to the maximum bin or bins; and
calculating if the frequency of the at least one peripheral bin and the maximum bin or bins exceeds a further threshold to determine if only the maximum bin or bins are selected or the at least one peripheral bin is selected together with the maximum bin or bins for deriving Rᵣₑᵥ.

16. A method according to claim 1, wherein R is calculated based on
$$R = \frac{[ \frac{1}{{PD}_{response ( red )}} ] [ \frac{{AC}_{red}}{{DC}_{red}} ]}{[ \frac{1}{{PD}_{response ( IR )}} ] [ \frac{{AC}_{IR}}{{DC}_{IR}} ]}$$
where,
PD_{response(red)} is a response factor of the first light illumination, wherein the first light illumination is red light;
PD_{response(IR)} is a response factor of the second light illumination, wherein the second light illumination is infrared light;
AC_{red} is an AC component value of the red light;
DC_{red} is a DC component value of the red light;
AC_{IR} is an AC component value of the infrared light; and
DC_{IR} is a DC component value of the infrared light.

17. A method according to claim 1, wherein SpO2 is derived from the revised ratio Rᵣₑᵥ based on:
$$\frac{( a - b \times {R}_{rev} )}{( m - n \times {R}_{rev} )}$$
where
a=ε_{Hb}(λ_{R})
b=ε_{Hb}(λ_{IR})
m=ε_{Hb}(λ_{R})−ε_{HbO}_{₂}(λ_{R})
n=ε_{Hb}(λ_{IR})−ε_{HbO}_{₂}(λ_{IR})
and where,
ε_{Hb}(λ_{R}) is an extinction coefficient of hemoglobin at Red wavelength;
ε_{Hb}(λ_{IR}) is an extinction coefficient of hemoglobin at IR wavelength;
ε_{HbO}_{₂}(λ_{R}) is an extinction coefficient of oxyhemoglobin at Red wavelength, and
ε_{HbO}_{₂}(λ_{IR}) is extinction coefficient of oxyhemoglobin at IR wavelength.

18. (canceled)

19. A method of determining SpO2 of a subject from an optical measurement, the method comprising:
(i) in a first interval, obtaining a first signal from a first light illumination and a second signal from second light illumination, the second light illumination having a wavelength which is different from that of the first light illumination;
(ii) calculating values of a ratio R from the first and second signals;
(iii) in a second interval, obtaining an ambient signal with the first and second light illuminations switched off;
(iv) obtaining AC_{amb} and DC_{amb};
(v) calculating a ratio, R_{amb-signal}, based on AC_{amb} and AC_{signal};
where
AC_{amb} is an AC component value of the ambient signal;
DC_{amb} is a DC component value of the ambient signal;
AC_{signal} is an AC component value of the first or second signal;
wherein, the method further comprises
(vi) providing a zoning schema divided into a plurality of reference zones with each reference zone defined by respective values of first, second and third reference variables which are associated respectively with R, R_{amb-signal} and DC_{amb}; each reference zone being associated with a corresponding method of computing SpO2;
(vii) comparing R, R_{amb-signal} and DC_{amb} with respective reference variables of the zoning schema to determine which one of the plurality of reference zones is a selected zone; and
(viii) applying the method of computing SpO2 corresponding to the selected zone for determining SpO2.

20. A method according to claim 19, further comprising obtaining a revised ratio, Rᵣₑᵥ based on R and using Rᵣₑᵥ in place of R at step (vii).

21. A method according to claim 20, further comprising pairing the first and second signals in which an amplitude of each cardiac rhythm cycle of the first signal is aligned to an amplitude of a respective cardiac rhythm cycle of the second signal to form a plurality of paired windows.

22. A method according to claim 21, further comprising,
for each paired window, calculating values of the ratio R from the paired first and second signals;
based on the calculated R values, binning the calculated R values into predetermined frequency bins; and
selecting at least one of the frequency bins to derive the revised ratio, Rᵣₑᵥ.

23. (canceled)

24. A method of determining SpO2 of a subject from an optical measurement, the method comprising:
(i) in a first interval, obtaining a first signal from a first light illumination and obtaining a second signal from a second light illumination, the second light illumination having a wavelength which is different from that of the first light illumination;
(ii) pairing the first and second signals in which an amplitude of each cardiac rhythm cycle of the first signal is aligned to an amplitude of a respective cardiac rhythm cycle of the second signal to form a plurality of paired windows;
(iii) for each paired window, calculating values of a ratio R from the paired first and second signals;
(iv) based on the calculated R values, binning the calculated R values into predetermined frequency bins; and
(v) selecting at least one of the frequency bins to derive a revised ratio, Rᵣₑᵥ;
(vi) in a second interval, obtaining an ambient signal with the first and second light illuminations switched off;
(vii) obtaining AC_{amb} and DC_{amb};
(viii) calculating a ratio, R_{amb-signal}, based on AC_{amb} and AC_{signal};
where
AC_{amb} is an AC component value of the ambient signal;
DC_{amb} is a DC component value of the ambient signal;
AC_{signal} is an AC component value of the first or second signal;
wherein, the method further comprises
(viiii) providing a zoning schema divided into a plurality of reference zones with each reference zone defined by respective values of first, second and third reference variables which are associated respectively with Rᵣₑᵥ, R_{amb-signal} and DC_{amb}; each reference zone being associated with a corresponding method of computing SpO2;
(x) comparing Rᵣₑᵥ, R_{amb-signal} and DC_{amb} with respective reference variables of the zoning schema to determine which one of the plurality of reference zones is a selected zone; and
(xi) applying the method of computing SpO2 corresponding to the selected zone for determining SpO2.

25. (canceled)

26. Optical measurement apparatus for determining SpO2 of a subject, the apparatus comprising:
(a) a photodetector configured to
(i) in a first interval, obtain a first signal from a first light illumination and a second signal from second light illumination, the second light illumination having a wavelength which is different from that of the first light illumination;
(ii) in a second interval, obtain an ambient signal with the first and second light illuminations switched off;
(b) a processor configured to:
(iii) calculate values of a ratio R from the first and second signals;
(iv) obtain AC_{amb} and DC_{amb};
(v) calculate a ratio, R_{amb-signal}, based on AC_{amb} and AC_{signal};
where
AC_{amb} is an AC component value of the ambient signal;
DC_{amb} is a DC component value of the ambient signal;
AC_{signal} is an AC component value of the first or second signal; and
(c) a zoning schema divided into a plurality of reference zones with each reference zone defined by respective values of first, second and third reference variables which are associated respectively with R, R_{amb-signal} and DC_{amb}; each reference zone being associated with a corresponding method of computing SpO2;
wherein the processor is further configured to:
(vi) compare R, R_{amb-signal} and DC_{amb} with respective reference variables of the zoning schema to determine which one of the plurality of reference zones is a selected zone; and
(vii) apply the method of computing SpO2 corresponding to the selected zone for determining SpO2.
